(19) United States
(12) Patent Application Publication
(10) Pub. No.: US 2014/0228232 A1
(43) Pub. Date: Aug. 14, 2014

(54) IN VITRO METHOD FOR PREDICTING SEMEN FERTILITY

(71) Applicants: Sara Larriba Bartolomé, Barcelona (ES); Lluís Bassas Arnau, Badalona (ES)

(72) Inventors: Sara Larriba Bartolomé, Barcelona (ES); Lluís Bassas Arnau, Badalona (ES)

(73) Assignees: FUNDACIO PUIGVERT, Barcelona (ES); INSTIUT D' INVESTIGACIÓ BIOMÈDICA DE BELLVITGE (IDBELL), l' Hopitalet de Llobregat (ES)

(21) Appl. No.: 14/124,484

(22) PCT Filed: Apr. 23, 2012
(86) PCT No.: PCT/ES2012/070268

§ 371 Date: Feb. 25, 2014

(30) Foreign Application Priority Data
Jun. 6, 2011 (EP) 11382187.0

(51) Int. Cl. C12Q 1/68

(52) U.S. Cl. CPC C12Q 1/6883
USPC 506/9; 435/6.12

(57) ABSTRACT

In vitro method for predicting fecundity of semen. The present invention belongs to the field of reproductive medicine, particularly related to male and female infertility. Thus, the present invention is focused on the evaluation and assessment of the sperm expression gene profile, in order to identify a specific group of genes whose expression profile is correlated with the fecundity ability of spermatozoa. This group of genes could be used as biomarkers for discriminating those samples with the worst ability to fertilize oocyte.

### FIELD OF THE INVENTION

The present invention may be included in the field of the medicine in general, more particularly in the field of reproductive medicine which comprises the study of the processes involved in reproduction, particularly male and female infertility.

Thus the present invention is focused on the evaluation and assessment of the sperm expression gene profile, in order to identify a specific group of genes whose expression profile is correlated with the fecundity ability of spermatozoa. This group of genes could be used as biomarkers for discriminating those samples with the worst ability to fertilize oocyte.

### STATE OF THE ART

Traditionally, selection of sperm donors has relied on microscopic assessment to determine semen quality. The assessment of male fertility is based on the descriptive information provided by the basic semen analysis, including sperm count, motility and morphology. New threshold values for semen parameters have been recently updated (Cooper et al., 2010) using men who had produced recent pregnancy as reference individuals. However, these basic semen parameters do not reflect the sperm function and their clinical value in predicting fertility is questionable. Although there is a clear correlation between the semen quality and the probability of conception (Guzick et al., 2001), the wide overlap of parameters between fertile and infertile men suggest that this basic semen analysis (mainly based on sperm count, motility and morphology) does not always correlate with a successful fecundity, and seriously hampers the diagnostic power of semen analysis (Bartoov et al., 1993). The significant proportion of couples having unexplained infertility, in spite of the good quality of the semen, suggests that abnormal sperm function can be due to molecular defects in some cases (Lewis, 2007). Many efforts have been devoted to build up new diagnostic tests to provide more accurate information on the fecundity potential of human spermatozoa (Samplaski et al., 2010) but none of them have yet met the requirements to be adopted for clinical purposes.

Spermatozoa contain, besides the haploid genetic material, an abundant number of functionally viable transcripts (Krawetz, 2005). SAGE and cDNA microarrays on spermatozoa have identified between 3000-7000 different transcripts (Ostermeier et al., 2002; Zhao et al., 2006), commonly considered as remnants of stored mRNA from post-meiotically active genes reflecting the accurate development of spermatogenesis (review in (Miller & Ostermeier, 2006)). The potential for an active post-meiotic production of transcripts, however, exists: a persistence of a low but detectable level of transcription in mature sperm cells was later described (Miteva et al., 1995) and more recently it has been by detection of transcriptional and translational activities, determined in human sperm during capacitation and acrosome reaction (Gur & Breitbart, 2006; Naz, 1998). Moreover, these stored mRNAs were suggested to be used during the first steps of fertilization, which are not sensitive to transcriptional inhibitors, and then contribute to the paternal printing (Braude et al., 1988; Siffroi & Dadoune, 2001). Ostermeier et al (Ostermeier et al., 2004) reported for the first time that human spermatozoa can deliver mRNA to the oocyte during fertilization. Some of these mRNAs have been described to be translated de novo in oocyte after fertilization, thus supporting the hypothesis that, at least some transcripts, might have a function during or beyond the process of fertilization (Gur & Breitbart, 2006) and additionally contribute to the early transcriptome of the embryo (Boerke et al., 2007).

Sperm mRNAs present in the ejaculated spermatozoa have been suggested to represent a genetic fingerprint, and could be considered a historical record of what happened in gene expression during spermatogenesis (Zhao et al., 2006). Some studies have reported differences in the amount of certain sperm transcripts between different groups of men. A decreased PRM1/PRM2 ratio (Steger et al., 2008) as well as PSG1 and HLA-E mRNA levels (Avendano et al., 2009) were observed in infertile compared to fertile men. The DEAD box polypeptide 4 (VASA) mRNA expression was higher in normozoospermic than in oligozoospermic men (Guo et al., 2007). A different expression signature or fingerprint was also determined (related to the differences of sperm motility) between normozoospermic and teratozoospermic men (Platts et al., 2007). Interestingly, differences in expression of a few hundreds of transcripts between fertile and infertile men with normal semen parameters using microarray analysis have been recently described (Garrido et al., 2009).

Assisted reproduction techniques (ART) have revolutionized the treatment of infertile couples. Among them, therapeutic donor insemination (TDI) provides an ideal first approach to achieve pregnancy in couples with a clear severe male infertility factor. After artificial insemination treatment with sperm donors, differences in pregnancy rates are observed among those donors, although all of them present normal parameters of semen quality. Additional studies suggest that some characteristics of the thawed samples used in the insemination, such as morphometry, number of motile sperm, curvilinear velocity, average path velocity or straight line velocity, show good correlation with pregnancy rates after IUI (intrauterine insemination) (Macleod & Irvine, 1995; Marshburn et al., 1992), although their use in diagnosis has not been validated. The TDI programme represents, thus, an adequate model to study male subfertility of unknown etiology.

Bearing in mind the state of the art, it seems that an accurate method to predict the fecundity of semen has not yet found. This method would also serve to confirm the results obtained by the other known methods which, as cited above, are not definitive, for example the basic semen analysis including sperm count, motility and morphology.

The success of ICSI (Intracytoplasmic Sperm Injection) for men with male infertility has been one of the reasons for the delay in implementing appropriated markers of sperm function. Any sperm, even those which would not normally have the capacity to fertilize, are injected into oocytes bypassing selection barriers. In order to treat couples with the least invasive treatment, better prognostic tests for fertile sperm, fertile oocytes and viable embryos are needed to improve the outcome of the treatment and obtain higher pregnancy rates. The findings of the present invention contribute to the search and selection of the most valuable gene markers potentially useful as additional tools for predicting and/or improving the success of assisted reproduction techniques leading to increase the pregnancy outcome of said techniques. The present invention shows an expression fingerprint (profile) related to the fecundity ability of sperm that could complement the semen analysis carried out by conventional fertility tests. By means of the method of the present invention it is possible to select those samples from donor semen banks with appropriate conditions to be used for assisted reproduction techniques, as well as giving realistic information about the chances of success of conjugal assisted reproduction techniques to those couples with unexplained infertility.

### DESCRIPTION OF THE INVENTION

The present invention is focused on the evaluation and assessment of the sperm expression gene profile, in order to define a specific group of genes whose expression is indicative of the fecundity ability of spermatozoa. The expression values of this group of genes could be used as biomarkers to predict fecundity ability of men with normal semen parameters, therefore complementing the results obtained by the other known fertility assessment methods which, as cited above, are not definitive.

In spite of having the normal parameters of semen quality, differences in the fecundity of donor sperm are observed after insemination therapies (Marshburn et al., 1992). Classical sperm variables at the time of baseline evaluation of donors have limited value to predict their reproductive fitness. Thus, close supervision of the clinical results obtained for each donor is the only pragmatic way to discard those who show poor pregnancy rates after a reasonable number of insemination cycles (Johnston et al., 1994). Thus, the aim of the present invention is to define a specific gene expression profile able to reflect the fecundity ability of a given semen donor with better efficiency than classical or conventional semen quality parameters.

To address this issue, a cohort of semen donors with a good semen quality and with a detailed record of reproductive outcome preferably using IUI insemination in different female receptors, were studied. Recruitment of semen donors was done among young university students, who had unknown fertility status at the time of donation, thus being representative of the normozoospermic general population. Therefore, the method of the invention is suitable to investigate the molecular features of unexplained male infertility, because it circumvents some of the shortcomings present in infertile couples, such as the confusing role of the significant proportion of female causes that also contribute to reproductive failure.

Therefore the first step of the present invention is focused on searching and finding out the most valuable gene markers, among an initial panel of 87 target genes described to be expressed in human spermatozoa (Ostermeier et al., 2002; Zhao et al., 2006), which can be efficiently used for predicting and/or improving the pregnancy outcome. Consequently, a molecular classifier of the fertility status of semen donors, preferably for IUI, based on gene expression profiles of sperm has been developed in the present invention. The panel of biomarker genes was selected in a training series of semen donors with a detailed record of IUI reproductive outcome in different female receptors, thus obviating the confounding role of female to reproductive failure. The potential of the gene panel of the present invention as a predictive classifier was validated in an independent series of donors.

As may be seen in the examples below, it was found that the gene expression profile of the genes EIF5A, RPL13, RPL23A, RPS27A, RPS3, RPS8 and TOMM7, either taken separately or in combination, can be efficiently used as a predictive tool for discriminating samples with worst pregnancy rate (PR), showing better results than that obtained from the combination of traditional/conventional sperm quality parameters. In a particularly preferred embodiment of the present invention, a multiplex model was defined with the group of four genes consisting of: EIF5A, RPL13, RPL23A and RPS27A, selected from the group of seven genes cited above.

Therefore, a molecular classifier of the fertility status of semen donors, particularly for IUI, may be developed based on the expression fingerprint of the above cited seven genes, particularly based on the expression profile of said selection group of four genes. Overall, this model has 97% sensitivity and 90% specifity as a predictive tool for discriminating samples with IUI pregnancy rate<13.6%, far better than that obtained from the assessment bases on the combination of traditional sperm quality parameters. Moreover, the method of the invention could complement the semen conventional analysis of well known fertility tests, in order to select those samples from donor semen banks with appropriate conditions to be used for assisted reproduction, as well as to suggest either a conjugal or a donor IUI in those couples with unexplained infertility.

Sensitivity and specificity are statistical measures of the performance of a binary classification test, also known in statistics as classification function. Sensitivity measures the proportion of actual positives which are correctly identified as such. Specificity measures the proportion of negatives which are correctly identified. A theoretical, optimal prediction should reach 100% sensitivity and 100% specificity. So, the method of the invention showing 97% sensitivity and 90% specificity should be considered as a strong and solid tool for predicting fecundity ability of spermatozoa.

Real time PCR amplification in 384-well TaqMan® Low Density Arrays (TLDAs) was considered as an appropriate method for implementing the present invention and for future potential diagnosis purposes, because it allows the simultaneous quantitative amplification of 48 reactions with a minimum cDNA material and a reduced variability due to pipetting. Moreover, in order to improve experimental accuracy, data were normalized to suitable reference genes, which showed constitutive and stable expression levels in the samples investigated.

Therefore the first embodiment of the present invention refers to an in vitro method for predicting fecundity ability of spermatozoa comprising the expression profile analysis of at least one of the following seven genes: EIF5A, RPL13, RPL23A, RPS27A, RPS3, RPS8 or TOMM7, or combinations thereof. In a preferred embodiment the method of the invention comprises the expression profile analysis of a selection group of genes comprising at least one of the following four genes: EIF5A, RPL13, RPL23A or RPS27A, or combinations thereof. Moreover, in a particularly preferred embodiment, the present invention comprises the expression profile analysis of the group of genes consisting of: EIF5A, RPL13, RPL23A and RPS27A.

The method of the invention is carried out taking into account that the genes EIF5A, RPL13, RPL23A, RPS27A, RPS3, RPS8 or TOMM7 are infra-expressed in samples with low fecundity ability as compared with their expression level in samples with optimal fecundity ability (see FIG. 3).

The method of the invention is particularly focused on predicting fecundity ability of spermatozoa when intrauterine insemination is selected as fertilization technique. However, it is equally suitable to be used when an alternative reproduction technique is used.

Finally, the method of the invention is able to predict fecundity ability both in semen donors from semen banks and in couples with unexplained infertility. Therefore, the method of the invention could complement the semen conventional analysis, as fertility test, for example in order to select those samples from donor semen banks with appropriate conditions to be used for assisted reproduction as well as to suggest a conjugal or a donor TUT in those couples with unexplained infertility.

Consequently, following the above explained method of the invention it would be possible to improve the pregnancy outcome.

Since the above cited seven genes are well conserved in different species, the method of the invention may be used to predict fecundity ability not only of human spermatozoa but also of animal spermatozoa. The prediction of fecundity of animal spermatozoa is interesting in several fields, for example in order to control the fecundity of animals in danger of extinction.

### BRIEF DESCRIPTION OF THE FIGURES

FIG. 1. Flow chart representing the number of samples and candidate genes through the stages for phase I (exploring phase) and II (validating phase).

FIG. 2. Expression ratios of the differentially expressed genes (Kruskal-Wallis test) in IUI semen samples with worse pregnancy rate (<15.70%, group 1, black bars), intermediate (15.71-23%, group 2, striated bars) and those with better pregnancy rate (>23.01%, group 3, white bars) using RPS17 as normalizer. Significant differences between group 1 or 2 and group 3 (controls) are indicated by asterisks (*, p≦0.05; **, p≦0.01; ***, p≦0.001). Data with IC 95%.

FIG. 3. Semen EIF5A, RPL13, RPL23A, RPS27A, RPS3, RPS8 and TOMM7 expression ratio as a marker for fecundity of sperm. Horizontal line indicates the transcript ratio threshold value (EIF5A: 0.67; RPL13: 0.65; RPL23A: 0.85; RPS27A: 1.01, RPS3: 0.58, RPS8: 0.60 and TOMM7: 0.58) that predicts an IUI pregnancy rate>13.5% (receiver operating characteristic curve analysis). The sensitivity and specificity values are described in Table 5.

FIG. 4.

A. Distribution of samples by their IUI pregnancy rate and the predicted probability of the four genetic variables, showing the potential of the model to discriminate samples with worse fertility ability.

B. Distribution of samples by their IUI pregnancy rate and the predicted probability of the four clinical variables, showing that this model is unable to discriminate samples with worse fertility ability.

### EXAMPLES

### Example 1

### Experimental Design

The study was divided into two phases. In the first phase or training phase, a general overview of gene expression behaviour was determined in relation to the pregnancy rates obtained by sperm donors and a gene set expression signature was obtained. In the second phase, the gene set fingerprints was validated as a predictive diagnostic tool in an independent series of donor semen samples. The study was approved by the Institutional Review Board of the Centre.

### Example 2

### Selection of Semen Donors

Cryopreserved semen samples (0.5 ml straws) were procured from the semen bank made up of semen donors referred to the Andrology Service of the Fundació Puigvert and used for TDI from 1994 to 2006. Advertisement for candidate donors was done among universitary students, and most of them had not attempted procreation at the time of recruitment. The clinical procedures for screening semen donors included full personal and familiar medical history to rule out heritable conditions, physical examination and a minimum of two semen tests [performed in accordance with World Health Organization guidelines (WHO, 1999) except for motility assessments, that were done at room temperature]. Spermiograms included volume, pH, sperm concentration, motility, vitality, morphology and antisperm antibodies. Computer assisted sperm analysis (CASA) was performed with a Hamilton-Thorn 2030 system (software version 6.4). Serological tests for HIV I and II, hepatitis B and C, cytomegalovirus and syphilis were done at baseline. At the end of the donations, after six months of quarantine, only donors that tested negative were used. Karyotype was done in donors enrolled after the year 2000.

All semen samples were frozen within two hours after collection, on an equal volume of glycerol-egg-yolk-citrate cryopreservative medium (Sperm Freezing medium, Irvine Scientific, Santa Ana, Calif., USA) in liquid nitrogen, using 1.8 mL cryovials, and stored at −196° C. until needed. Cryosurvival was assessed as the percent progressive motility of sperm after thawing in a bath at 37° C.

### Example 3

### Female Receptors and Insemination Procedures

Women entering into the program of TDI (Therapeutic Donor Insemination) of the Fundació Puigvert that were inseminated with samples of the selected donors were considered for the study. Indications for TDI included severe male factors in the majority of cases, and ejaculatory disturbances or hereditary conditions of the husband. Ovulatory status was studied by biphasic temperature charts and progesterone at midluteal phase, and a normal hysterosalpingography.

In all cases mild follicular stimulation was induced with 75 UI/day of gonadotrophins (Neo-Fertinorm or Pergonal, Serono SA, Spain), and monitored by analysis of estradiol and transvaginal ultrasonography. Ovulation was induced by 10000 UI of HCG (Profasi, HCG Lepori) when at least one follicle of >18 mm was observed. Thawed semen samples (0.5 mL) were diluted with 2 mL of Hams F-10 medium with 0.5% HSA and prepared by differential centrifugation using density gradients (Puregon). Final volume was adjusted to 0.4 mL. Inseminations were done on two consecutive days after 24 and 48 hours after administration of HCG (Human Chorionic Gonadotropin) using an insemination catheter (#4220, Gynétics Medical, Lommel, Belgium). If B-HCG levels were increased 2 to 4 weeks after the inseminations, pregnancy was confirmed by ultrasound scan. Selection of semen donors for insemination was performed by the medical staff on the basis of a matching phenotype of the husband. Semen donor was changed, after 2 or 3 insemination cycles to a particular woman, if pregnancy had not occurred. Donors failing to produce pregnancies were eventually discarded for further use after 20-25 cycles of treatment.

### Example 4

### Selection of Semen Samples

A total of 68 samples from normozoospermic donors were recruited in the present invention. The inclusion criteria were as follows: having at least 4 surplus frozen aliquots available after the use for insemination purposes, average sperm concentration≧40 millions/mL; A+B motility≧30%, normal morphology≧7% at the time of initial assessment, and >10 insemination cycles per sperm donor.

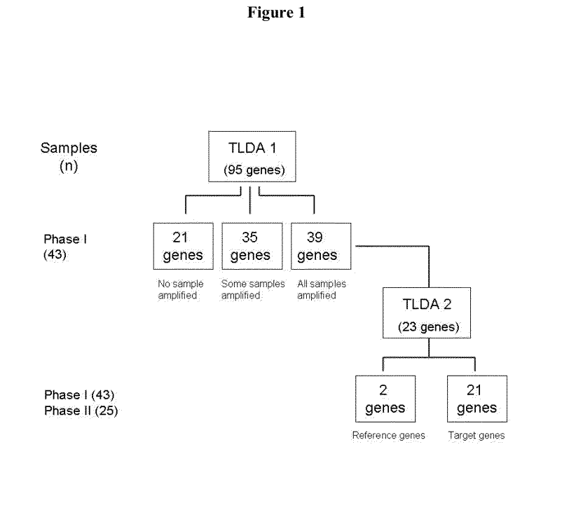

In the first phase, approximately ⅔ of samples (n=43) were randomly chosen for gene expression analysis (FIG. 1). Samples were initially classified into three groups (tertiles) that differed in their ability to produce pregnancy after TDI: samples with the worse pregnancy rate (PR) (group 1), intermediate (group 2) and those that achieved the best PR (group 3, as control of a successful PR) (Table 1). Semen samples among IUI groups presented similar features on the basis of the concentration, motility and morphology of spermatozoa (p=0.116; p=0.814 and p=0.936 respectively), as well as, the motility post-thaw of spermatozoa (p=0.245) also considered for analysis (Table 1.A) (semen conventional parameters). Other clinical factors from male and receptor female related with pregnancy outcome are also indicated (Table 1.B).

TABLE 1. A

| | | Semen | | | Normal | Motility |
|---|---|---|---|---|---|---|
| IUI (>10 | | volume | Sperm conc. | Motility | morphology | post-thaw |
| cycles/sample) | n | (mL) | (million/mL) | (%) | (%) | (%) |
| Group 1 | 14 | 3.50 ± 0.51 | 79.21 ± 19.28 | 59.17 ± 5.99 | 23.53 ± 5.50 | 33.71 ± 5.52 |
| (≦15.70% pr) | | | | | | |
| Group 2 (15.71-23.00% | 14 | 3.82 ± 0.43 | 101.75 ± 20.93 | 58.35 ± 5.41 | 26.30 ± 6.03 | 38.33 ± 5.44 |
| pr) | | | | | | |
| Group 3 | 15 | 3.71 ± 0.75 | 87.23 ± 17.62 | 57.06 ± 5.33 | 24.60 ± 4.47 | 39.95 ± 4.62 |
| (≧23.01% pr) | | | | | | |
| Total | 43 | | | | | |

IUI: intrauterine insemination.
pr: pregnancy rate.
n: number of donor samples.
conc: concentration.

TABLE 1. B

| | | | | | | | Receptor | |
|---|---|---|---|---|---|---|---|---|
| | | | | | | | women | |
| | | | Male | Receptor | | Female | without | |
| | | Male | Body | women | Female | Body | GPD per | |
| IUI (>10 | | age | Mass | per donor | age | Mass | donor | Abortion |
| cycles/sample) | n | (years) | index | (n) | (years) | index | (%) | rate (%) |
| Group 1 | 14 | 24.57 ± 2.53 | 23.03 ± 1.29 | 14.50 ± 4.37 | 34.69 ± 0.91 | 25.13 ± 1.07 | 72.64 ± 6.95 | 12.56 ± 14.20 |
| (≦15.70% pr) | | | | | | | | |
| Group 2 (15.71-23.00% | 14 | 25.71 ± 2.63 | 22.93 ± 1.25 | 14.29 ± 3.40 | 35.40 ± 0.75 | 24.64 ± 0.68 | 76.21 ± 7.12 | 11.58 ± 6.95 |
| pr) | | | | | | | | |
| Group 3 | 15 | 25.53 ± 2.63 | 23.23 ± 1.25 | 12.93 ± 2.70 | 34.79 ± 0.53 | 25.39 ± 0.80 | 75.93 ± 5.85 | 15.63 ± 5.66 |
| (≧23.01% pr) | | | | | | | | |
| Total | 43 | | | | | | | |

IUI: intrauterine insemination.
pr: pregnancy rate.
n: number of donor samples.
GPD: gyneocological pathology diagnosis.

In the second phase, gene expression fingerprint was validated as a predictive diagnostic tool on 25 additional samples (FIG. 1).

### Example 5

### RNA Extraction and cDNA Synthesis

In order to enrich fertile spermatozoa and remove somatic contaminants from gene expression analysis of sperm, the frozen-thawed semen samples were individually purified by centrifugation through discontinuous Percoll gradient (65%-90%). Such centrifugation technique is routinely used in ART and relies on the fractioning of cells according to their velocity of sedimentation (Gandini et al., 1999). Semen samples (0.5 mL) were layered on top of the 65% gradient layer and further processed by centrifugation at 175×g for 30 minutes. This results in the formation of a pellet fraction largely constituted by sperm with normal morphology, and in a supernatant interphase fraction enriched in defective germ cells (sperm with morphological abnormalities, as well as other immature cell types) (Chen & Bongso, 1999; Gil-Guzman et al., 2001). The obtained pellet was resuspended in supplemented HamF10 medium and processed again by centrifugation at 400×g for 5 minutes.

Total RNA was obtained from the Percoll-purified spermatozoa using NucleoSpin® RNA II Kit (Macherey-Nagel, Duren, Germany), according to the instructions provided by the manufacturer with minor modifications. Briefly, lysis buffer was added to the samples at 600 μl/10⁷ cells. The lysates were homogenized with a 20-gauge needle and heated for 30 min at 60° C. Then it was proceeded to step 4 of the kit, including a DNAse digestion step. RNA was eluted in 40 μl of RNAse-free water. Sample purity and RNA integrity was assessed by reverse-transcription (RT)-PCR using PRM2 primers as previously described (Ostermeier et al., 2005).

For gene expression study, single-stranded cDNA was obtained by RT of 200 ng of RNA, using random primers and MultiScribe™ Reverse Transcriptase from the High Capacity cDNA Reverse Transcription Kit (AB, Foster City, Calif., USA) at 37° C. for 120 min. Two independent RT reactions were performed from each RNA sample. The resulting cDNA solution was stored at −20° C. until use.

### Example 6

### Gene Expression Quantification

Quantitative real-time PCR assays were performed by PCR arrays on micro fluidic cards (MFC), using 384-well TaqMan® Low Density Arrays (TLDAs) on an Applied Biosystems 7900HT Fast Real-Time PCR System (AB, Foster City, Calif., USA). Half of RT-reaction was applied on each port, each one connecting to 48 reaction wells. A first approach (TLDA1) (FIG. 1) was performed on the 96-gene format MFC (95 experimental assays and 1 TLDA amplification control) allowing simultaneous measurement of 87 target genes that were selected based on human spermatozoa cell location from cDNA microarrays (Ostermeier et al., 2002; Zhao et al., 2006), and 8 ubiquitously expressed genes, as potential reference genes. Genes and the corresponding assays on demand used for the setup of the TLDA are listed in table 2.

TABLE 2

| Gene Symbol | Gene Name | Assay ID | Role |
|---|---|---|---|
| PRM-1+ | Protamine-1 | Hs00358158_g1 | Nuclear |
| | | | condensation |
| PRM-2+ | Protamine-2 | Hs00172518_m1 | Nuclear |
| | | | condensation |
| RERE+ | arginine-glutamic acid | Hs00201558_m1 | Transcriptional |
| | dipeptide (RE) repeats | | regulation |
| FOXG1/FOXG1B+ | Forck head box G1B | Hs01850784_s1 | Transcription |
| | | | regulation. (Early |
| | | | embryo patterning) |
| TEAD1/TEF1 | TEA domain family member | Hs00744253_s1 | Transcription |
| | 1 (SV40 transcriptional | | regulation. (Early |
| | enhancer factor). | | embryo patterning) |
| | Transcriptional enhancer | | |
| | factor TEF-1 | | |
| RBM9+ | RNA binding motif protein 9 | Hs00329214_s1 | Transcriptional |
| | | | regulation (RNA |
| | | | binding related) |
| LRRFIP1 | Leucine rich repeat | Hs00190993_m1 | Transcriptional |
| | interacting protein 1 | | regulation (RNA |
| | | | binding related) |
| IREB2 | Iron-responsive element | Hs00386293_m1 | Transcriptional and |
| | binding protein 2 | | translational |
| | | | regulation (RNA |
| | | | and protein binding |
| | | | related) |
| EIF3G | Eukaryotic translation | Hs00186772_m1 | Translational |
| | initiation factor 3 delta | | regulation, |
| | subunit | | initiation |
| EIF3J/EIF3SI | Eukaryotic translation | Hs00825842_g1 | Translational |
| | initiation factor 3, subunit J | | regulation, |
| | (subunit I) | | initiation |
| EIF3M/GA17 | Eukaryotic translation | Hs00272235_m1 | Translational |
| | initiation factor 3, subunit M | | regulation, |
| | | | initiation. |
| | | | Fusogenic protein |
| | | | (Sperm-oocyte |
| | | | interaction?) |
| EIF5A+ | Eukaryotic translation | Hs00744729_s1 | Translational |
| | initiation factor 5A | | regulation, |
| | | | initiation |
| EIF5 | Eukaryotic translation | Hs00820472_m1 | Translational |
| | initiation factor 5 | | regulation, |
| | | | initiation |
| RPS3+ | Ribosomal protein S3 | Hs02385124_g1 | Protein biosynthesis |
| | | | (ribosomal protein). |
| | | | DNA repair |
| RPS6+ | Ribosomal protein S6 | Hs02339423_g1 | Protein biosynthesis |
| | | | (ribosomal protein) |
| RPS8+ | Ribosomal protein S8 | Hs01374307_g1 | Protein biosynthesis |
| | | | (ribosomal protein) |
| RPS13+ | ribosomal protein S13 | Hs01945436_u1 | Protein biosynthesis |
| | | | (ribosomal protein) |
| RPS16 | Ribosomal protein S16 | Hs01598518_gH | Protein biosynthesis |
| | | | (ribosomal protein) |
| RPS17# | Ribosomal protein S17 | Hs00734303_g1 | Protein biosynthesis |
| | | | (ribosomal protein) |
| RPS18+ | Ribosomal protein S18 | Hs02387368_g1 | Protein biosynthesis |
| | | | (ribosomal protein) |
| RPS26 | Ribosomal protein S26 | Hs00955682_g1 | Protein biosynthesis |
| | | | (ribosomal protein) |
| RPS27+ | Ribosomal protein S27 | Hs01378332_g1 | Protein biosynthesis |
| | | | (ribosomal protein) |
| RPS27A/S27a+ | Ribosomal protein S27a | Hs01923841_uH | Protein biosynthesis |
| | | | (ribosomal protein) |
| RPS29+ | Ribosomal protein S29 | Hs03004310_g1 | Protein biosynthesis |
| | | | (ribosomal protein) |
| RPL4+ | Ribosomal protein L4 | Hs03044647_g1 | Protein biosynthesis |
| | | | (ribosomal protein) |
| RPL5 | Ribosomal protein L5 | Hs00851991_u1 | Protein biosynthesis |
| | | | (ribosomal protein) |
| RPL7+ | Ribosomal protein L7 | Hs02596927_g1 | Protein biosynthesis |
| | | | (ribosomal protein) |
| RPL10A+ | Ribosomal protein L10a | Hs01912344_uH | Protein biosynthesis |
| | | | (ribosomal protein) |
| RPL13+ | Ribosomal protein L13 | Hs00761672_s1 | Protein biosynthesis |
| | | | (ribosomal protein) |
| RPL17 | Ribosomal protein L17, | Hs01597859_m1 | Protein biosynthesis |
| | transcript variant 2 | | (ribosomal protein) |
| RPL17 | Ribosomal protein L17, | Hs00748900_s1 | Protein biosynthesis |
| | transcript variant 1 | | (ribosomal protein) |
| RPL23A+ | Ribosomal protein L23a | Hs01921329_g1 | Protein biosynthesis |
| | | | (ribosomal protein) |
| RPL24 | Ribosomal protein L24 | Hs02338570_gH | Protein biosynthesis |
| | | | (ribosomal protein) |
| RPL27A | Ribosomal protein L27a | Hs00741143_s1 | Protein biosynthesis |
| | | | (ribosomal protein) |
| RPL29# | Ribosomal protein L29 | Hs00988959_gH | Protein biosynthesis |
| | | | (ribosomal protein) |
| RPL30 | Ribosomal protein L30 | Hs00265497_m1 | Protein biosynthesis |
| | | | (ribosomal protein) |
| RPL35 | Ribosomal protein L35 | Hs00855441_gH | Protein biosynthesis |
| | | | (ribosomal protein) |
| RPLP2 | Ribosomal protein, large, P2 | Hs01115130_g1 | Protein biosynthesis |
| | | | (ribosomal protein) |
| FAU | Finkel-Biskis-Reilly murine | Hs00609872_g1 | Protein biosynthesis |
| | sarcoma virus (FBR-MuSV) | | (ribosomal protein) |
| | ubiquitously expressed | | |
| RSL1D1 | Ribosomal L1 domain | Hs00378363_g1 | Protein biosynthesis |
| | containing 1 | | (ribosomal protein) |
| EEF2 | Eukaryotic translation | Hs01012839_g1 | Protein biosynthesis |
| | elongation factor 2 | | (ribosomal protein) |
| RPS4Y1 | 40S ribosomal protein S4, Y | Hs00606158_m1 | Protein biosynthesis |
| | isoform (Y-linked 1) | | (ribosomal protein) |
| MRPL40 | Mitochondrial ribosomal | Hs00186843_m1 | Protein biosynthesis |
| | protein L40 | | (mitochondrial |
| | | | ribosomal protein) |
| MRPS18B | Mitochondrial ribosomal | Hs00204096_m1 | Protein biosynthesis |
| | protein S18B | | (mitochondrial |
| | | | ribosomal protein) |
| FARSB | Phenylalanyl-tRNA | Hs00271714_m1 | Protein biosynthesis |
| | synthetase beta chain | | (cytoplasmic |
| | cytoplasmic | | protein) |
| COPS5 | COP9 constitutive | Hs00272789_m1 | Protein biosynthesis |
| | photomorphogenic homolog | | (nuclear protein) |
| | subunit 5 (Arabidopsis) | | |
| RPS6KA2 | Ribosomal protein S6 kinase | Hs00179731_m1 | Protein amino acid |
| | | | modification |
| | | | (phosphorylation) |
| ST6GALNAC4 | ST6 (alpha-N-acetyl- | Hs00205241_m1 | Protein amino acid |
| | neuraminyl-2,3-beta- | | modification |
| | galactosyl-1,3)-N- | | (glycosilation in |
| | acetylgalactosaminide alpha- | | Golgi apparatus) |
| | 2,6-sialyltransferase 4 | | |
| NARS | Asparaginyl-tRNA synthetase | Hs00189846_m1 | Protein binding |
| | | | related (aspartyl- |
| | | | tRNA |
| | | | aminoacylation) |
| QARS | Glutaminyl-tRNA synthetase | Hs00192530_m1 | Protein binding |
| | | | related (glutaminyl- |
| | | | tRNA |
| | | | aminoacylation) |
| UBC | Ubiquitin C | Hs00824723_m1 | Protein amino acid |
| | | | modification |
| | | | (Ubiquitin |
| | | | conjugating system) |
| TMED2/RNP24 | Transmembrane emp24 | Hs00607277_m1 | Protein |
| | domain trafficking protein 2/ | | transportation |
| | Homo sapiens coated vesicle | | (cytoplasm protein) |
| | membrane protein | | |
| SCAMP1 | Secretory carrier membrane | Hs00792736_m1 | Protein |
| | protein 1 | | transportation |
| | | | (cytoplasm protein) |
| VTI1B | Vesicle transport through | Hs00762282_s1 | Protein |
| | interaction with t-SNAREs | | transportation |
| | homolog 1B (yeast) | | (cytoplasm protein) |
| SLC29A2 | Solute carrier family 29 | Hs00155426_m1 | Protein |
| | (nucleoside transporters), | | transportation |
| | member 2 | | (cytoplasm protein) |
| SLC25A19 | Solute carrier family 25 | Hs00222265_m1 | Protein |
| | (mitochondrial thiamine | | transportation |
| | pyrophosphate carrier), | | (mytochondrial |
| | member 19, variant 2 | | protein) |
| SLC25A25 | Solute carrier family 25 | Hs01595832_m1 | Protein |
| | (mitochondrial carrier; | | transportation |
| | phosphate carrier), member | | (mytochondrial |
| | 25 | | protein) |
| SLC25A39 | Solute carrier family 25, | Hs00255405_m1 | Protein |
| | member 39 | | transportation |
| | | | (mytochondrial |
| | | | protein) |
| TOMM7+ | Translocase of outer | Hs01628668_s1 | Protein |
| | mitochondrial membrane 7 | | transportation |
| | homolog (yeast) | | (mytochondrial |
| | | | protein) |
| SFXN3 | Sideroflexin 3 | Hs00229616_m1 | Protein |
| | | | transportation |
| | | | (mytochondrial |
| | | | protein) |
| CSE1L | CSE1 chromosome | Hs00354853_m1 | Protein-nucleus |
| | segregation 1-like (yeast) | | import (nuclear |
| | | | pore complex) |
| IPO5 | Importin 5 | Hs00267008_m1 | Protein-nucleus |
| | | | import (nuclear |
| | | | pore complex) |
| XPO1 | Exportin 1 (CRM1 homolog, | Hs00185645_m1 | Protein-nucleus |
| | yeast) | | import (nuclear |
| | | | pore complex) |
| XPO7 | Exportin 7 | Hs00209262_m1 | Protein-nucleus |
| | | | import (nuclear |
| | | | pore complex) |
| KPNA2 | Karyopherin alpha 2 (RAG | Hs00818252_g1 | Protein-nucleus |
| | cohort 1, importin alpha 1) | | import (nuclear |
| | | | pore complex) |
| RANBP2 | RAN binding protein 2 | Hs00397898_g1 | Protein-nucleus |
| | | | import (nuclear |
| | | | pore complex) |
| PDIA3 | Protein disulfide isomerase | Hs00607126_m1 | Protein folding |
| | family A, member 3 | | (endoplasmic |
| | | | reticulum) |
| WBSCR21/ABHD11 | Abhydrolase domain | Hs00541033_g1 | Catalytic activity |
| | containing 11 | | (metabolic feature |
| | | | of spermatozoa?) |
| RNF144B/IBRDC2 | Ring finger 144B/IBR damin | Hs00403456_m1 | Catalytic activity, |
| | containing 2 | | protein |
| | | | ubiquitination |
| | | | (metabolic feature |
| | | | of spermatozoa?) |
| CCNB1IP1 | Cyclin B1 interacting protein | Hs00820463_g1 | Catalytic activity |
| | 1 | | (metabolic feature |
| | | | of spermatozoa?) |
| ENO1+ | Enolase 1 (alpha) | Hs00361415_m1 | Catalytic activity |
| | | | (metabolic feature |
| | | | of spermatozoa?) |
| COX5B | Cytochrome c oxidase subunit | Hs00426948_m1 | Catalytic activity |
| | Vb | | (metabolic feature |
| | | | of spermatozoa?) |
| FADS1 | Fatty acid desaturase 1 | Hs00203685_m1 | Catalytic activity |
| | | | (metabolic feature |
| | | | of spermatozoa?) |
| AKAP-4 | A kinase (PRKA) anchor | Hs00275849_m1 | Signal transduction. |
| | protein 4 | | Involved in sperm |
| | | | motility |
| | | | (fertilization and |
| | | | activation of |
| | | | oocyte) |
| FGFR1 | Fibroblast growth factor | Hs00241111_m1 | Signal transduction |
| | receptor 1 | | (spermatogenesis |
| | | | and fertilization?) |
| TM4SF6 | Tetraspanin 6 | Hs00170288_m1 | Signal transduction |
| | | | (spermatogenesis |
| | | | and fertilization?) |
| GRIN2C | Glutamate receptor, | Hs01016626_m1 | Signal transduction |
| | ionotropic, N-methyl D- | | (spermatogenesis |
| | aspartate 2C | | and fertilization?) |
| IL6ST | Interleukin 6 signal transducer | Hs00174360_m1 | Signal transduction |
| | | | (spermatogenesis |
| | | | and fertilization?) |
| VAV2 | Vav 2 oncogene | Hs00610104_m1 | Signal transduction |
| | | | (spermatogenesis |
| | | | and fertilization?) |
| WNT5A | Wingless-type MMTV | Hs00998537_m1 | Signal transduction. |
| | integration site family, | | Embryonic |
| | member 5A | | development, |
| | | | Cellular |
| | | | differentiation and |
| | | | morphometric |
| | | | patterning |
| HLA-E | Major histocompatibility | Hs00428366_m1 | Signal transduction. |
| | complex, class 1, E | | Immune response |
| | | | protein Signal |
| | | | transducer protein |
| | | | (spermatogenesis |
| | | | and fertilization?) |
| eNOS/NOS3 | Endothelial nitric oxide | Hs00167166_m1 | Capacitation |
| | synthase, nitric oxide synthase | | |
| | 3 (endothelial cell) | | |
| nNOS/NOS1 | Neuronal nitric oxide | Hs00167223_m1 | Capacitation |
| | synthase, nitric oxide synthase | | |
| | 1 (neuronal) | | |
| CLGN | Calmegin precursor variant 1 | Hs00189073_m1 | Testis-specific |
| | | | endoplasmic |
| | | | reticulum |
| | | | chaperone protein. |
| | | | (Sperm-egg |
| | | | interaction, |
| | | | Fertilization) |
| CLU | Clusterin | Hs00156548_m1 | Protein binding. |
| | | | Cell-cell |
| | | | interations; cellular |
| | | | processes for |
| | | | embryo |
| | | | development |
| PTHLH | Parathyroid hormone-like | Hs00174969_m1 | Regulation of gene |
| | hormone | | expression. |
| | | | Hormonal |
| | | | activity.(Pregnancy) |
| CRHBP | Corticotropin releasing | Hs00181810_m1 | Signal transducer |
| | hormone binding protein | | protein. Hormonal |
| | | | activity. |
| | | | (Pregnancy) |
| HPRT | Hypoxanthine | Hs99999909_m1 | |
| | phosphoribosyltransferase 1 | | |
| | (Lesch-Nyhan syndrome) | | |
| HMBS | Hydroxymethylbilane | Hs00609297_m1 | |
| | synthase | | |
| PPIA | Cyclophilin A, peptidylprolyl | Hs99999904_m1 | |
| | isomerase A | | |
| PGM1 | Phosphoglucomutase 1 | Hs00160062_m1 | |
| GUSB | Glucuronidase, beta | Hs99999908_m1 | |
| PGK1 | Phosphoglycerate kinase 1 | Hs99999906_m1 | |
| TBP | TATA box binding protein | Hs00427620_m1 | |
| KIAA0999/L19 | KIAA0999 protein | Hs00228549_m1 | |

Gene symbols in bold and underlined depict those genes that showed positive PCR-amplifications in all samples. Those genes included in TLDA2 are indicated with + (target genes) or # (reference genes) symbols.

A subsequent second approach (TLDA2) (FIG. 1) was performed on the 24-gene format MFC, which included 21 target genes, 2 reference genes and an amplification control (Table 2). TLDA2 comprised a different RT reaction of the same sample on TLDA1 approach.

Patient and control group samples were always analysed as paired samples in the same analytical run in order to exclude between-run variations. Real-time PCR data were pre-processed and stored in SDS 2.2 software (AB, Foster City, Calif., USA).

To confirm reproducibility and precision of real-time PCR experiments, inter-assay variation of samples amplified on both approaches were determined. Variation was measured as the coefficient of variation (CV) of Ct from the Ct mean value of both TLDA approaches. In the above mentioned RT-PCR runs, inter-assay variation ranged from 0.63% to 1.60% with the exception of PRMI (2.40%), PRM2 (2.04%), ENO1 (4.13%) and RERE (3.42%), confirming high reproducibility and precision for most of the 23 genes included in the TLDA1 and TLDA2 approaches.

### Example 7

### Data Analysis

All statistical analyses were performed using the SPSS version 12 (Lead Technologies, Chicago, USA) software.

The nonparametric Kruskal-Wallis test was first used to analyze the differences in clinical data among IUI study groups of phase I.

Selection of the reference gene/s, showing the most stable expression and the lowest variation among samples, was calculated with both the GeNorm (Vandesompele et al., 2002) and the SPSS version 12 (Lead Technologies, Chicago, USA) programs.

Differences in absolute expression levels of reference genes among study groups 1, 2 and 3 were analyzed by the Kruskal-Wallis test. In order to select the target genes to be included in TLDA2 experiment, differences in absolute expression of TLDA1 target genes in group 1 compared to group 3 were evaluated by the nonparametric Mann-Whitney U test.

Raw data normalization was performed with the qBase program (Hellemans et al., 2007) by using one reference gene as well as by applying geometric averaging of two reference genes, in parallel. Relative quantification (RQ) values were expressed using the 2^{-ΔΔCt} method as fold changes in the target gene normalized to the reference gene and related to the expression of a control sample. In the phase I, the mean value of the TLDA1 and TLDA2 normalized 2^{-ΔΔCt} values for each sample were then subjected to evaluation of statistical significance of differential expression among groups. The nonparametric Kruskal-Wallis test was used to analyze the differences in relative expression of target genes among the IUI study groups. Mann-Whitney U test was used to evaluate differences in relative expression of target genes in patient group 1 or 2 compared to group 3.

Pearson product moment correlation coefficients were calculated to determine the correlation between the expression ratios of the target genes and the IUI PR. Receiver operating characteristic (ROC) curve analysis of the relative expression values was used for distinguishing those individuals with PR≦13.6% for IUI. Accuracy was measured as the area under the ROC curve (AUC). The threshold value was determined by Youden's index, calculated as sensitivity plus specificity −1 (Skendzel & Youden, 1970).

Multivariate binary logistic regressions were used for selection of the optimal combination of genes associated with fertilization status of the phase I samples and for validating the combination of genes as a predictive tool in samples of phase II. A backward stepwise (Conditional) method was used to drop insignificant terms. The multivariate regression model included the genes found to significantly distinguish IUI-PR≦13.6%. The binary logistic regression model provides the following estimation of the logit function: Logit(p)=B0+B1X1+B2X2+ . . . .

where p=P (adequate fertility potential for insemination), Logit(p)=log(p/(1−p))=log(Odds), B=log OR and Xn=the expression value of the selected genes. Therefore, if we use this estimated model as a prediction model, with the standard classification cutoff of 0.5 (that is, we predict an individual as “having adequate fertility potential for insemination” if its estimated fecundations probability is greater than 0.5), we would classify individuals with a positive Logit function estimation as “adequate for insemination” and individuals with negative Logit function estimation as “inadequate for insemination”.

Binary logistic regressions of a single genetic variant as well as single/combination of clinical parameters were calculated for comparison of predictive values of the model.

A p-value<0.05 was considered significant.

### Example 8

### Selection of Genes for the TLDA2 Approach

The presence of mRNA for 74 out of the 95 genes of the TLDA1 study (genes in bold Table 2) was confirmed by RT-PCR in human ejaculated spermatozoa. The rest of genes (n=21) could not be amplified (Ct value>33) under the conditions of the study suggesting that the transcript levels were underneath the detection threshold of the technique. From the 74 genes amplified, 35 were excluded for further analysis due to poor amplification efficiency across samples (missing expression values>80%). The mRNA levels of genes amplified in all samples of the study (Table 2) were further evaluated (n=39) (FIG. 1).

In order to achieve precise and reliable quantitative expression results of the genes under study, measurement of gene expression by real-time RT-PCR requires at least one proper internal control gene for normalization purposes. None of the eight genes previously described as ubiquitously expressed have quantifiable expression values, so they were excluded as normalizers. Therefore, among the 39 remaining genes, those showing stable expression levels in the samples investigated were selected and used as normalizers or reference genes. For this purpose, Genorm program was used and this program selected the RPS17 and RPL29 as the most stably expressed genes (M=0.038 for both genes). Moreover, it was assessed that these genes were not differentially expressed among the groups of the study (p>0.05). These two genes were included in the TLDA2 approach as reference genes.

The selection of target genes included in the TLDA2 approach (Table 2) was performed taking into account those genes that presented statistical difference in Ct values between IUI group 1 and 3.

### Example 9

### Relative Gene Expression Profile of Donor Sperm

Once the TLDA1 and TLDA2 Ct data from the phase I samples were obtained, the quantification of the 21 target gene mRNA levels was, thus, expressed as relative transcript levels using RPS17 as a single reference gene as well as RPS17 and RPL29 genes combination as reference value for both TLDA experiments.

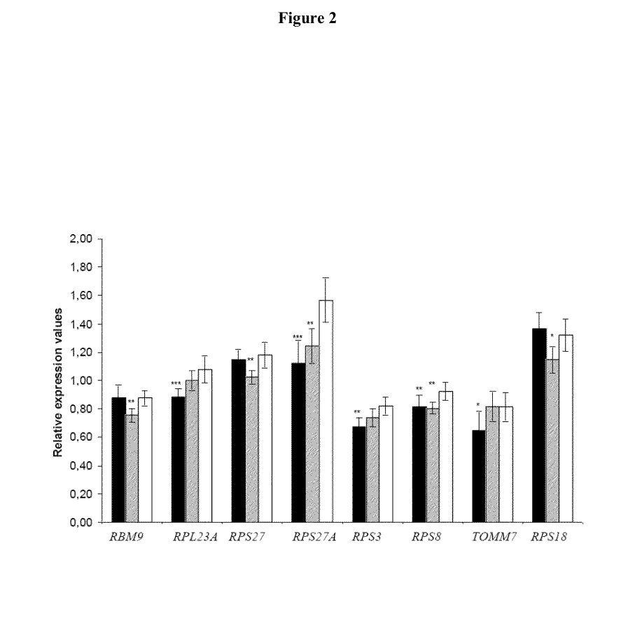

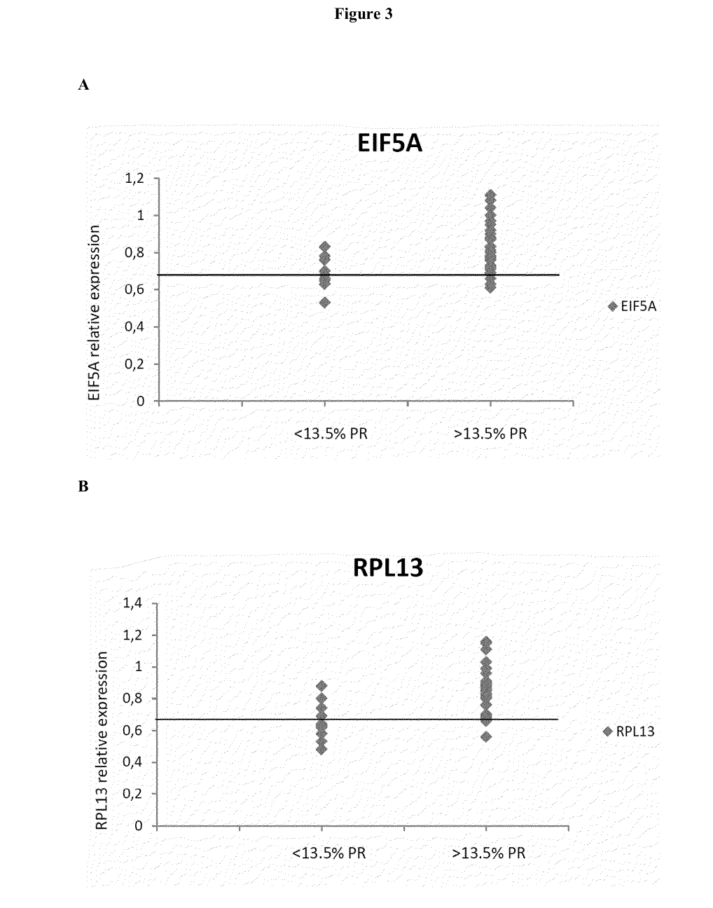

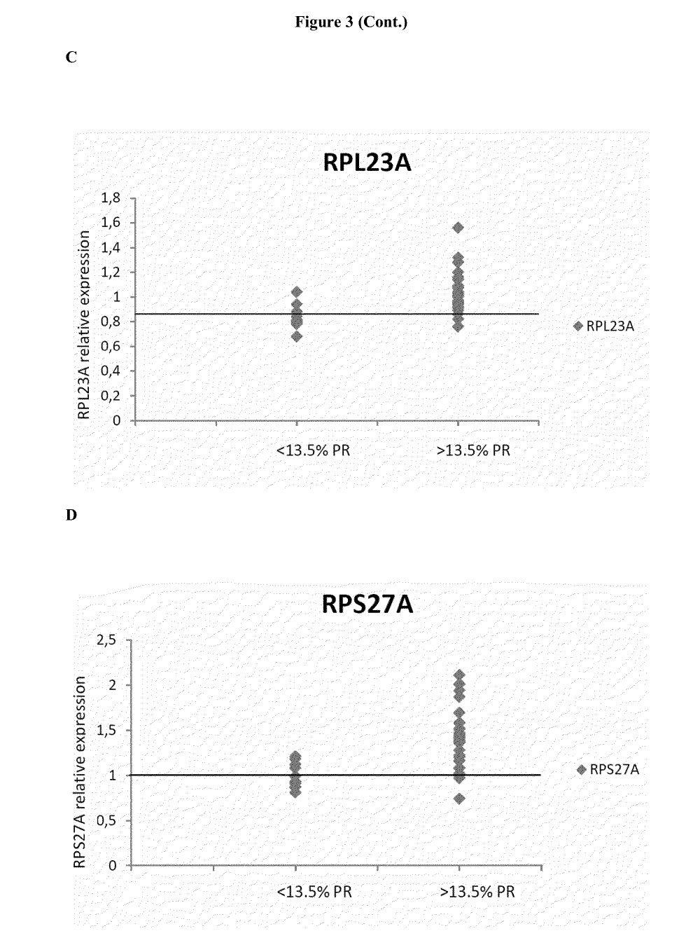

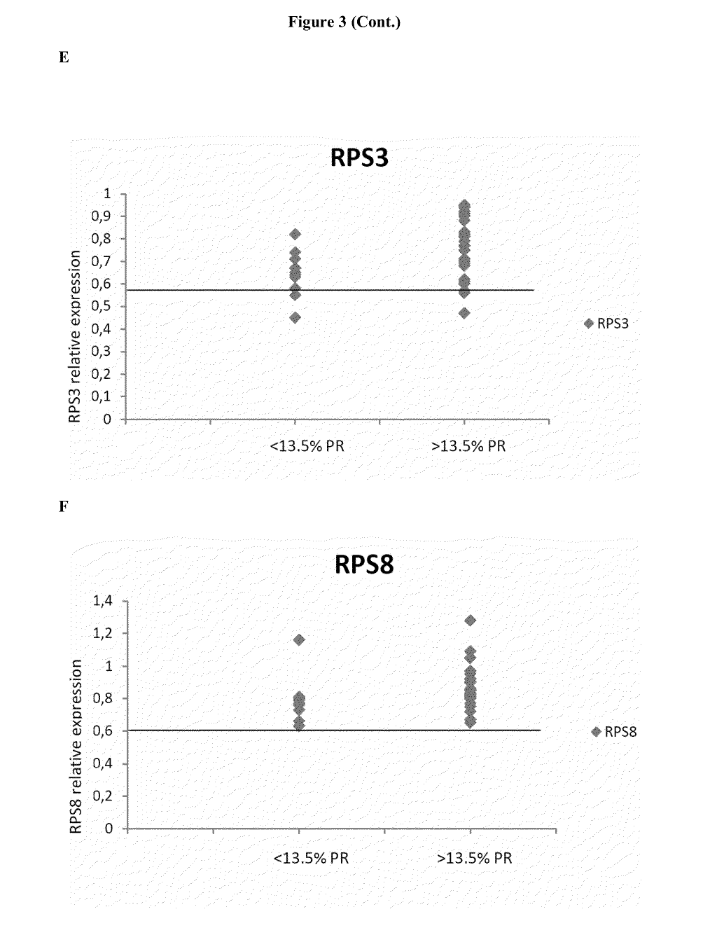

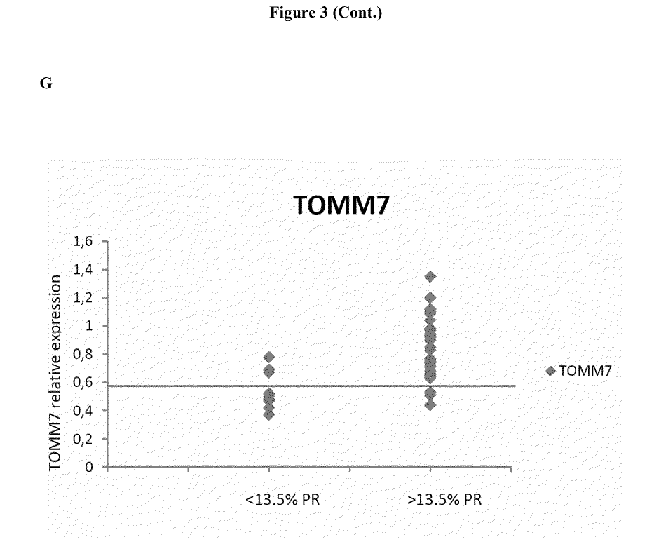

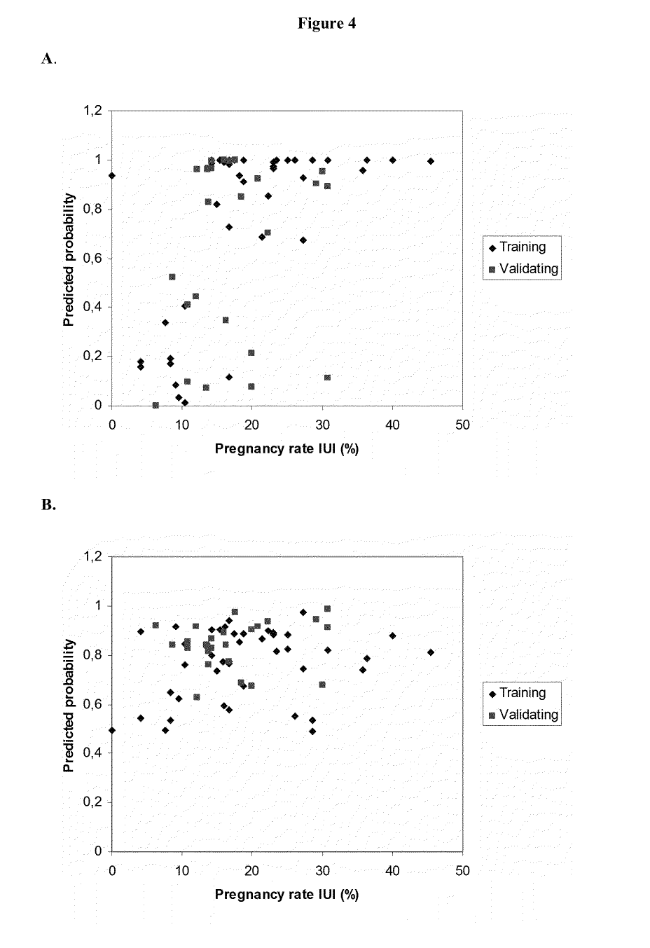

When samples were classified taking into account the PR that resulted when used in IUI, a group comprising eight differentially expressed genes were found among the three groups: RPL23A, RPS27A, RPS8 (p≦0.01), RBM9, RPS27, RPS3, TOMM7 and RPS18 (p≦0.05), when normalized with both, single and combination, of reference genes (FIG. 2). All of them presented small intra-group standard deviation values (0.08-0.30).

A selection group of genes comprising: RPL23A, RPS27A, RPS3, RPS8 and TOMM7 genes showed a significant fold-change decrease in Group 1 of 1.22, 1.39, 1.22, 1.13, and 1.26 respectively, when compared to Group 3 (p≦0.05).

As both, single and combination, of reference genes, data normalization resulted in the same statistical result, gene expression data normalized with RPS17 was subsequently used for giving more simplicity to the model.

### Example 10

### Correlation Study Between Gene Expression Profiles and Semen Conventional Fecundity Parameters or Pregnancy Rates

No significant correlation was found between the sperm concentration or motility semen parameters and the relative mRNA expression levels of any of the 21 genes analysed. However morphology of spermatozoa was found to be positively correlated with FOXG1 (r:0.341; p=0.025) and RPS8 (r:0.371; p=0.014) transcript levels.

In order to assess whether there is an association between gene expression and the assisted reproduction PR and to confirm whether the results could be of physiological relevance, a correlation study was performed between the normalized gene expression ratios and the PR mean value of the insemination cycles in which the sample was used. Significant positive correlation coefficients were found between IUI PR and the transcription levels of six genes: RPL23A, RPL4, RPS27A, RPS3, RPS8 and TOMM7 (p<0.05). Table 3 shows the Pearson correlation coefficients and adjusted p-values (r; p) between the expression ratios of the target genes and the pregnancy rates after assisted reproduction for all the samples analyzed. Correlation between sperm clinical parameters and pregnancy rates are also shown for comparison. Significant differences (p≦0.05) are indicated in bold. PR: pregnancy rate. IUI: intrauterine insemination.

TABLE 3

| | |
|---|---|
| IUI PR | Abortion rate |

Gene expression

| | | |
|---|---|---|
| RPL23A | 0.467; p = 0.002 | 0.110; p = 0.483 |
| RPL4 | 0.419; p = 0.005 | 0.055; p = 0.726 |
| RPS27A | 0.530; p = 0.000 | 0.206; p = 0.185 |
| RPS3 | 0.463; p = 0.002 | 0.103; p = 0.509 |
| RPS8 | 0.450; p = 0.002 | −0.014; p = 0.930 |
| TOMM7 | 0.308; p = 0.044 | 0.189; p = 0.225 |
| RPL10A | 0.283; p = 0.066 | 0.403; p = 0.007 |
| RPS6 | 0.086; p = 0.584 | 0.326; p = 0.033 |
| RBM9 | 0.145; p = 0.352 | −0.301; p = 0.050 |

Sperm clinical parameters

| | | |
|---|---|---|
| Sperm concentration | 0.032; p = 0.837 | −0.043; p = 0.783 |
| A + B movility | −0.024; p = 0.878 | 0.050; p = 0.751 |
| Sperm morphology | 0.154; p = 0.323 | −0.035; p = 0.826 |

The same type of analysis was performed for other clinical parameters derived from the IUI, such as the viability rate and abortion rate. Three additional genes: RPL10A, RPS6 and RBM9 expression values were found to significantly correlate with abortion rate in IUI study (p≦0.05) (Table 3).

It is hypothesized that there is a threshold level of transcripts with the potential of discriminating those samples with the worst ability to fertilize oocyte. We selected as the state variable the 25th percentile of samples that is ≦13.6% of PR for IUI. The ROC curve analysis of gene expression levels in IUI resulted in good predictive accuracy (AUC>0.720) of the expression values of seven genes: EIF5A, RPL13, RPL23A, RPS27A, RPS3, RPS8 and TOMM7 (p<0.01). They were selected as potential genetic biomarkers of sperm fertility status. Table 4 shows a ROC analysis showing the predictive probabilities of clinical and genetic variables for discriminating those samples with worse fertility ability. IUI: pregnancy rates<13.6%. AUC: area under the curve. CI: confidence interval.

TABLE 4

| | Variables | | |
|---|---|---|---|
| IUI study | AUC | 95% CI | p-value |
| Semen concentration | 0.588 | 0.363-0.813 | 0.404 |
| Motility | 0.348 | 0.141-0.556 | 0.151 |
| Normal morphology | 0.615 | 0.399-0.831 | 0.275 |
| Motility post-thaw | 0.598 | 0.416-0.781 | 0.350 |
| EIF5A | 0.827 | 0.691-0.964 | 0.002 |
| RPL13 | 0.859 | 0.724-0.994 | 0.001 |
| RPL23A | 0.882 | 0.752-1.000 | 0.000 |
| RPS27A | 0.894 | 0.798-0.990 | 0.000 |
| RPS3 | 0.788 | 0.642-0.934 | 0.006 |
| RPS8 | 0.770 | 0.586-0.954 | 0.011 |
| TOMM7 | 0.870 | 0.748-0.992 | 0.000 |

Moreover, Table 5 shows a univariable logistic regression analyses, showing that each of the seven genes comprised in the Table 5 may be efficiently used to predict fecundity ability of spermatozoa (validated in samples of phase II). The specificity (Sp) and the sensitivity (Sn) for predicting an IUI PR≦13.6% is depicted for each gene.

TABLE 5

| | Variables | | |
|---|---|---|---|
| | Phase I | Phase II | |
| | Sp; Sn | Sp; Sn | p-value |
| EIF5A | 50; 91 | 43; 94.5 | 0.008 |
| RPL13 | 60; 97 | 0; 100 | 0.004 |
| RPL23A | 70; 94 | 14; 94.4 | 0.003 |
| RPS27A | 60; 91 | 43; 72 | 0.003 |
| RPS3 | 20; 91 | 14; 94.4 | 0.013 |
| RPS8 | 0; 100 | 14; 94.4 | 0.119 |
| TOMM7 | 70; 88 | 28; 83 | 0.004 |

### Example 11

### Multiplex Model: Multivariate Logistic Regression Analysis

To determine if a multiplex model could improve performance over single biomarkers, the previously selected genetic biomarkers were analyzed in a multivariate regression analysis. A backward stepwise (conditional) method was used to drop insignificant terms from the model in phase I samples.

Referring IUI study, this analysis resulted in a model that included EIF5A, RPL13, RPL23A and RPS27A genes (Table 6). Table 6 shows multivariate logistic regression analyses of genetic variables compared to clinical variables in the different phases of the study. IUI: intrauterine insemination. SC: sperm concentration. MT: motility. MPh: morphology. MT-C: Motility post-thaw Sp: specificity. Sn: sensibility. OR: odds ratio. CI: confidence interval. Note: For the multivariate analysis, a backward stepwise (Conditional) method was used to drop insignificant terms in phase I samples. The resulting classifier was used in phase II samples for validation.

TABLE 6

| | Variables | | | | |
|---|---|---|---|---|---|
| | Phase I | Phase II | | 95% CI | |
| IUI study | Sp; Sn | Sp; Sn | OR | for OR | p-value |
| EIF5A + | 90; 97 | 71.5; 78 | | | |
| RPL13 + | | | | | |
| RPL23A + | | | | | |
| RPS27A | | | | | |
| EIF5A | | | 1.176 | 0.918-1.505 | 0.200 |
| RPL13 | | | 0.907 | 0.720-1.143 | 0.410 |
| RPL23A | | | 1.164 | 0.975-1.390 | 0.093 |
| RPS27A | | | 1.113 | 1.018-1.217 | 0.018 |
| SC + MT + | 20; 100 | 14; 94 | | | |
| MPh + MT-C | | | | | |
| SC | | | 1.018 | 0.987-1.051 | 0.259 |
| MT | | | 0.870 | 0.764-0.990 | 0.035 |
| MPh | | | 1.028 | 0.936-1.129 | 0.560 |
| MT-C | | | 1.095 | 0.987-1.215 | 0.087 |

The sensitivity and the specificity for predicting an IUI PR≦13.6% were 97% and 90%, respectively. The accuracy of the test was corroborated as the calculated AUC was 0.955 (p=0.000) and the p-value of Hosmer and Lemeshow test was 0.554. As comparison, the combination of the four clinical values of sperm resulted in a sensitivity of 100% but a specificity of 20% (AUC:0.761; p=0.013).

The classifier based on gene expression values of phase I samples was validated in samples of phase II, resulting in a sensitivity of 78% and a specificity of 71.5% (Table 6). For this sample distribution the classification of semen donors is predicted by the formula:

Log-odds (individual)=B0+B1X1+B2X2+B3X3+B4X4

If the log-odds of the individual results in a negative number, then the sample is classified as having inadequate fertility potential for IUI, if is positive it is classified as adequate.

### REFERENCES

Avendano, C., Franchi, A., Jones, E. & Oehninger, S. (2009) Pregnancy-specific {beta}-1-glycoprotein 1 and human leukocyte antigen-E mRNA in human sperm: differential expression in fertile and infertile men and evidence of a possible functional role during early development. Hum Reprod, 24, 270-277.

Bartoov, B., Eltes, F., Pansky, M., Lederman, H., Caspi, E. & Soffer Y. (1993) Estimating fertility potential via semen analysis data. Hum Reprod. 8, 65-70.

Boerke, A., Dieleman, S. J. & Gadella, B. M. (2007) A possible role for sperm RNA in early embryo development. Theriogenology, 68 Suppl 1, S147-155.

Braude, P., Bolton, V. & Moore, S. (1988) Human gene expression first occurs between the four- and eight-cell stages of preimplantation development. Nature, 332, 459-461.

Cooper, T. G., Noonan, E., von Eckardstein, S., Auger, J., Baker, H. W., Behre, H. M., Haugen, T. B., Kruger, T., Wang, C., Mbizvo, M. T. & Vogelsong, K. M. (2010) World Health Organization reference values for human semen characteristics. Hum Reprod Update, 16, 231-245.

Chen, M. J. & Bongso, A. (1999) Comparative evaluation of two density gradient preparations for sperm separation for medically assisted conception. Hum Reprod, 14, 759-764.

Gandini, L., Lenzi, A., Lombardo, F., Pacifici, R. & Dondero, F. (1999) Immature germ cell separation using a modified discontinuous Percoll gradient technique in human semen. Hum Reprod, 14, 1022-1027.

Garrido, N., Martinez-Conejero, J. A., Jauregui, J., Horcajadas, J. A., Simon, C., Remohi, J. & Meseguer, M. (2009) Microarray analysis in sperm from fertile and infertile men without basic sperm analysis abnormalities reveals a significantly different transcriptome. Fertil Steril, 91, 1307-1310.

Gil-Guzman, E., Ollero, M., Lopez, M. C., Sharma, R. K., Alvarez, J. G., Thomas, A. J., Jr. & Agarwal, A. (2001) Differential production of reactive oxygen species by subsets of human spermatozoa at different stages of maturation. Hum Reprod, 16, 1922-1930.

Guo, X., Gui, Y. T., Tang, A. F., Lu, L. H., Gao, X. & Cai, Z. M. (2007) Differential expression of VASA gene in ejaculated spermatozoa from normozoospermic men and patients with oligozoospermia. Asian J Androl, 9, 339-344.

Gur, Y. & Breitbart, H. (2006) Mammalian sperm translate nuclear-encoded proteins by mitochondrial-type ribosomes. Genes Dev, 20, 411-416.

Guzick, D. S., Overstreet, J. W., Factor-Litvak, P., Brazil, C. K., Nakajima, S. T., Coutifaris, C., Carson, S. A., Cisneros, P., Steinkampf, M. P., Hill, J. A., Xu, D. & Vogel, D. L. (2001) Sperm morphology, motility, and concentration in fertile and infertile men. N Engl J Med, 345, 1388-1393.

Hellemans, J., Mortier, G., De Paepe, A., Speleman, F. & Vandesompele, J. (2007) qBase relative quantification framework and software for management and automated analysis of real-time quantitative PCR data. Genome Biol, 8, R19.

Johnston, R. C., Kovacs, G. T., Lording, D. H. & Baker, H. W. (1994) Correlation of semen variables and pregnancy rates for donor insemination: a 15-year retrospective. Fertil Steril, 61, 355-359.

Krawetz, S. A. (2005) Paternal contribution: new insights and future challenges. Nat Rev Genet, 6, 633-642.

Lewis, S. E. (2007) Is sperm evaluation useful in predicting human fertility? Reproduction, 134, 31-40.

Macleod, I. C. & Irvine, D. S. (1995) The predictive value of computer-assisted semen analysis in the context of a donor insemination programme. Hum Reprod, 10, 580-586.

Marshburn, P. B., McIntire, D., Carr, B. R. & Byrd, W. (1992) Spermatozoal characteristics from fresh and frozen donor semen and their correlation with fertility outcome after intrauterine insemination. Fertil Steril, 58, 179-186.

Miller, D. & Ostermeier, G. C. (2006) Towards a better understanding of RNA carriage by ejaculate spermatozoa. Hum Reprod Update, 12, 757-767.

Miteva, K., Valkov, N., Goncharova-Peinoval, J., Kovachev, K., Zlatarev, S., Pironcheva, G. & Russev, G. (1995) Electron microscopic data for the presence of post-meiotic gene expression in isolated ram sperm chromatin. Cytobios, 83, 85-90.

Naz, R. K. (1998) Effect of actinomycin D and cycloheximide on human sperm function. Arch Androl, 41, 135-142.

Ostermeier, G. C., Dix, D. J., Miller, D., Khatri, P. & Krawetz, S. A. (2002) Spermatozoal RNA profiles of normal fertile men. Lancet, 360, 772-777.

Ostermeier, G. C., Goodrich, R. J., Diamond, M. P., Dix, D. J. & Krawetz, S. A. (2005) Toward using stable spermatozoal RNAs for prognostic assessment of male factor fertility. Fertil Steril, 83, 1687-1694.

Ostermeier, G. C., Miller, D., Huntriss, J. D., Diamond, M. P. & Krawetz, S. A. (2004) Reproductive biology: delivering spermatozoan RNA to the oocyte. Nature, 429, 154.

Platts, A. E., Dix, D. J., Chemes, H. E., Thompson, K. E., Goodrich, R., Rockett, J. C., Rawe, V. Y., Quintana, S., Diamond, M. P., Strader, L. F. & Krawetz, S. A. (2007) Success and failure in human spermatogenesis as revealed by teratozoospermic RNAs. Hum Mol Genet, 16, 763-773.

Samplaski, M. K., Agarwal, A., Sharma, R. & Sabanegh, E. (2010) New generation of diagnostic tests for infertility: review of specialized semen tests. Int J Urol, 17, 839-847.

Siffroi, J. P. & Dadoune, J. P. (2001) Accumulation of transcripts in the mature human sperm nucleus: implication of the haploid genome in a functional role. Ital J Anat Embryol, 106, 189-197.

Skendzel, L. P. & Youden, W. J. (1970) Systematic versus random error in laboratory surveys. Am J Clin Pathol, 54, 448-450.

Steger, K., Wilhelm, J., Konrad, L., Stalf, T., Greb, R., Diemer, T., Kliesch, S., Bergmann, M. & Weidner, W. (2008) Both protamine-1 to protamine-2 mRNA ratio and Bc12 mRNA content in testicular spermatids and ejaculated spermatozoa discriminate between fertile and infertile men. Hum Reprod, 23, 11-16.

Vandesompele, J., De Preter, K., Pattyn, F., Poppe, B., Van Roy, N., De Paepe, A. & Speleman, F. (2002) Accurate normalization of real-time quantitative RT-PCR data by geometric averaging of multiple internal control genes. Genome Biol, 3, RESEARCH0034.

World Health Organization (1999) Laboratory Manual for the Examination of Human Semen ans Sperm-Cervical Mucus Interaction, p. Cambridge University Press, New York.

Zhao, Y., Li, Q., Yao, C., Wang, Z., Zhou, Y., Wang, Y., Liu, L., Wang, Y., Wang, L. & Qiao, Z. (2006) Characterization and quantification of mRNA transcripts in ejaculated spermatozoa of fertile men by serial analysis of gene expression. Hum Reprod, 21, 1583-1590.

1. An in vitro method for predicting fecundity ability of spermatozoa, said method comprising analyzing the expression profile of at least one of the following genes: EIF5A, RPL13, RPL23A, RPS27A, RPS3, RPS8 or TOMM7, or combinations thereof,
wherein the genes EIF5A, RPL13, RPL23A, RPS27A, RPS3, RPS8 or TOMM7 are infra-expressed in samples with low fecundity ability as compared with their corresponding expression level thresholds in samples with optimal fecundity ability.

2. The in vitro method, according to claim 1, comprising analyzing the expression profile of at least one of the following genes: EIF5A, RPL13, RPL23A or RPS27A, or combinations thereof.

3. The in vitro method, according to claim 1, comprising analyzing the expression profile of the group of genes consisting of: EIF5A, RPL13, RPL23A and RPS27A.

4. (canceled)

5. The in vitro method, according to claim 1, wherein the fecundity ability of spermatozoa can be predicted when intrauterine insemination is elected as fertilization technique.

6. The in vitro method, according to claim 1, wherein the fecundity ability can be predicted both in semen donors from semen banks and in semen of men from couples with unexplained infertility.

7. The in vitro method, according to claim 1, wherein the fecundity ability can be predicted both in human and animal spermatozoa.

8. An in vitro method for improving pregnancy outcome, said method comprising predicting fecundity ability of spermatozoa using the method of claim 1.
